(19) United States
(12) Patent Application Publication
(10) Pub. No.: US 2025/0000865 A1
(43) Pub. Date: Jan. 2, 2025

(54) COMPOUND FOR REDUCING URIC ACID LEVELS

(71) Applicant: NEWSOARA BIOPHARMA CO., LTD., Shanghai (CN)

(72) Inventors: Yuannian LI, Shanghai (CN); Jinyu CHEN, Shanghai (CN); Qinghua ZHU, Shanghai (CN); Qian ZHU, Shanghai (CN)

(21) Appl. No.: 18/709,820

(22) PCT Filed: Nov. 11, 2022
(86) PCT No.: PCT/CN2022/131336

§ 371 Date: May 14, 2024

(30) Foreign Application Priority Data
Nov. 15, 2021 (CN) 202111345096.1

(51) Int. Cl. A61K 31/519; A61K 31/4196; A61K 31/426; A61P 13/02

(52) U.S. Cl. CPC A61K 31/519; A61K 31/4196; A61K 31/426; A61P 13/02

(57) ABSTRACT

The present invention provides use of a PDE4 inhibitor (e.g., compound of Formula (I)) or a pharmaceutically acceptable salt thereof in the manufacture of a medicament for reducing uric acid level, preventing an increased uric acid level in an individual, and treating, preventing, or alleviating a disease, disorder, or condition (for example, gout, hyperuricemia) associated with an increased uric acid level in an individual.

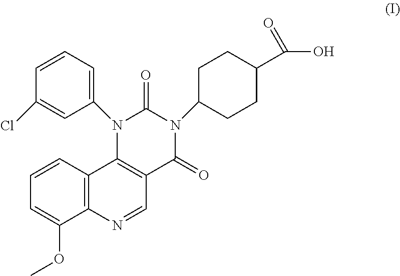 [SMILES: COc1cccc2c1ncc1c(=O)n(C3CCC(C(=O)O)CC3)c(=O)n(-c3cccc(Cl)c3)c12]

### FIELD OF THE INVENTION

The present application relates to the field of disease treatment, and particularly, to use of a PDE4 inhibitor (e.g., 4-[1-(3-chlorophenyl)-7-methoxy-2,4-dioxo-pyrimido[5,4-c]quinolin-3-yl]cyclohexanecarboxylic acid or a pharmaceutically acceptable salt thereof) in the manufacture of a medicament for reducing uric acid level, preventing an increased uric acid level, and treating, preventing, or alleviating a disease, disorder, or condition (for example, gout and hyperuricemia) associated with an increased uric acid level in an individual.

### BACKGROUND

Hyperuricemia is caused by excessive production or insufficient excretion of uric acid, and is considered as a factor inducing several diseases that seriously affect the quality of life. For example, hyperuricemia is considered as a factor inducing gout (the most common form of inflammatory arthritis) characterized by severe joint pain and tenderness caused by the crystallization and accumulation of urates.

Uricosuric agents and xanthine oxidase inhibitors are usually used to reduce the uric acid level and treat gout. The xanthine oxidase inhibitors (such as allopurinol or febuxostat) can reduce the formation of uric acid, and the uricosuric agents can inhibit the absorption of uric acid from the kidney back into the blood by URAT1 transporter. Benzbromarone is a uricosuric agent, which is known to be very effective in reducing serum uric acid (sUA). It is found that the treatment with benbromarone reduces sUA even after a single dose, and continues to reduce sUA after multiple doses, and can allow sUA to reach a target level of <6 mg/dL after long-term treatment. Unfortunately, similar to many other drugs, benbromarone may cause side effects of hepatotoxicity and acute liver failure.

There is still a need in the art for potent drugs for reducing uric acid with reduced toxicity.

### SUMMARY OF THE INVENTION

Inventors of the present application unexpectedly have found that PDE4 inhibitors are useful in reducing the uric acid level, preventing an increased uric acid level, and treating, preventing, or alleviating a disease, disorder, or condition associated with an increased uric acid level in an individual.

In an aspect, the present application provides use of a PDE4 inhibitor in the manufacture of a medicament for reducing uric acid level in an individual. In another aspect, the present application provides use of a PDE4 inhibitor in the manufacture of a medicament for preventing an increased uric acid level in an individual. In another aspect, the present application provides use of a PDE4 inhibitor in the manufacture of a medicament for treating, preventing, or alleviating a disease, disorder, or condition associated with an increased uric acid level in an individual.

In another aspect, the present application provides use of a compound of Formula (I) or a pharmaceutically acceptable salt thereof in the manufacture of a medicament for reducing uric acid level or preventing an increased uric acid level in an individual:

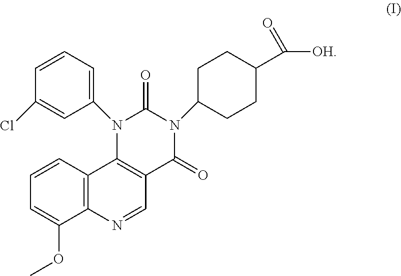 [SMILES: COc1cccc2c1ncc1c(=O)n(C3CCC(C(=O)O)CC3)c(=O)n(-c3cccc(Cl)c3)c12]

In another aspect, the present application provides use of a compound of Formula (I) or a pharmaceutically acceptable salt thereof in the manufacture of a medicament for treating, preventing, or alleviating a disease, disorder, or condition associated with an increased uric acid level in an individual.

In certain embodiments, the disease, disorder, or condition associated with an increased uric acid level described in the present application is selected from the group consisting of: gout, hyperuricemia, high uric acid level that does not reach the level usually diagnosed as hyperuricemia, renal dysfunction, urolithiasis, renal calculus, renal failure, diabetes, diabetes associated conditions, insulin resistance, metabolic syndrome, hypertension, hypothyroidism, hyperparathyroidism, obesity, inflammation, muscle spasm, local swelling, inflammation, joint pain, myocardial infarction, arthritis, tumor lysis syndrome, cognitive impairment, lead poisoning, psoriasis, sarcoidosis, non-alcoholic fatty liver disease, non-alcoholic fatty hepatitis, cardiovascular diseases, atherosclerosis, and transplants of blood, bone marrow or solid organs. In certain embodiments, the disease, disorder, or condition associated with an increased uric acid level described in the present application is gout or hyperuricemia. In certain embodiments, the disease, disorder, or condition associated with an increased uric acid level described in the present application is gout. In certain embodiments, the disease, disorder, or condition associated with an increased uric acid level described in the present application is hyperuricemia. In certain embodiments, the hyperuricemia described in the present application is primary hyperuricemia or secondary hyperuricemia. In certain embodiments, the secondary hyperuricemia described in the present application comprises hyperuricemia related to drugs or hyperuricemia related to various medical conditions.

In certain embodiments, the uric acid level described in the present application is the uric acid level in body fluids. In certain embodiments, the uric acid level described in the present application is the uric acid level in blood. In certain embodiments, the uric acid level described in the present application is the serum uric acid level.

In certain embodiments, the individual is further administrated one or more additional uric acid-reducing drugs. In certain embodiments, the additional uric acid-reducing drug is selected from the group consisting of: xanthine oxidase inhibitors, uricosuric agents, urate transporter-1 inhibitors, uricase and inhibin. In certain embodiments, the additional uric acid-reducing drug is selected from the group consisting of: allopurinol, febuxostat, lesinurad, benzbromarone, roflumilast, apremilast, sodium bicarbonate, potassium sodium hydrogen citrate, potassium sodium citrate, non-steroidal anti-inflammatory drugs, glucocorticoids and colchicine.

In certain embodiments, the individual described in the present application is a mammal. In certain embodiments, the individual described in the present application is human. In certain embodiments, before the medicament is administered to the individual, twice fasting serum uric acid levels in the individual on different days are higher than 420 μmol/L.

In certain embodiments, the medicament described in the present application is administered orally, subcutaneously, intravenously, intramuscularly or intraperitoneally. In certain embodiments, the medicament comprises no more than 5 mg, 10 mg, 15 mg, 20 mg, 25 mg, 30 mg, 35 mg, 40 mg, 45 mg, or 50 mg of the PDE4 inhibitor. In certain embodiments, the medicament comprises no more than 5 mg, 10 mg, 15 mg, 20 mg, 25 mg, 30 mg, 35 mg, 40 mg, 45 mg, or 50 mg of the compound of Formula (I) or a pharmaceutically acceptable salt thereof. In certain embodiments, the medicament is administered to the individual once a day, twice a day, or three times a day.

### BRIEF DESCRIPTION OF THE DRAWINGS

FIG. 1 shows the changes of uric acid levels in subjects in a 6 mg single-dose group and a 10 mg single-dose group (FIG. 1A) and 20 mg single-dose group and 30 mg single-dose group (FIG. 1B) of Compound I.

FIG. 2 shows the changes of uric acid levels in subjects in a 10 mg multi-dose group and a 20 mg multi-dose group of Compound I.

FIG. 3 shows the changes in uric acid levels from the baseline after the administration of Compound I once a day for consecutive 7 days at two different doses (10 mg and 20 mg), and the predicted results of changes in uric acid levels from the baseline after the administration of Compound I once a day for consecutive 7 days at other four different doses (6 mg, 15 mg, 30 mg, and 40 mg) with a PK/PD model. The prediction is performed based on the data from 54 healthy subjects, with typical values of population PK parameters and PD parameters. sUA represents serum uric acid.

FIG. 4 shows, available from the reference: Jacob Leander, Journal of Pharmacokinetics and Pharmacodynamics https://doi.org/10.1007/s10928-021-09747-y, the predicted results in changes of uric acid from the baseline after the administration of multiple doses of lesinurad (12 mg), febuxostat (80 mg) and a combination thereof. The prediction is performed based on 12 clinical test data from 434 subjects (including healthy subjects, patients with hyperuricemia and patients with renal damage). sUA represents serum uric acid.

### DETAILED DESCRIPTION

Phosphodiesterases (PDE) comprise a superfamily of enzymes responsible for the hydrolysis and inactivation of the second messengers cyclic adenosine monophosphate (cAMP) and cyclic guanosine monophosphate (cGMP). Eleven different PDE families (PDE1 to PDE11) have been identified so far, which differ in substrate preference, catalytic activity, sensitivity to endogenous activators and inhibitors, and coding genes.

The PDE4 isoenzyme family exhibits a high affinity for cAMP, but has only weak affinity for cGMP. Increased cAMP level caused by PDE4 inhibition are associated with the suppression of cell activation in a wide range of inflammatory and immune cells, including lymphocytes, macrophages, basophils, neutrophils, and eosinophils. PDE4 inhibitors with varied chemical structures have been disclosed in the art for treating and preventing chronic and acute inflammatory diseases.

Inventors of the present inventors unexpectedly have found that PDE4 inhibitors are useful in reducing the uric acid level, preventing an increased uric acid level, and treating, preventing, or alleviating a disease, disorder, or condition associated with an increased uric acid level in an individual.

### 1. Pharmaceutical Use of a PDE4 Inhibitor

In an aspect, the present application provides use of a PDE4 inhibitor in the manufacture of a medicament for reducing uric acid level in an individual. In another aspect, the present application provides use of a PDE4 inhibitor in the manufacture of a medicament for preventing an increased uric acid level. In another aspect, the present application provides use of a PDE4 inhibitor in the manufacture of a medicament for treating, preventing, or alleviating a disease, disorder, or condition associated with an increased uric acid level in an individual.

In the present application, the “PDE4 inhibitor” refers to an agent that can directly or indirectly reduce the level or activity of PDE4. In certain embodiments, the PDE4 inhibitor is a selective PDE4 inhibitor. For example, if the inhibitory activity of an agent on PDE enzymes other than PDE4 in the PDE superfamily is 1, 5, 10, 20, 30, 40, 50, 60, 70, 80, 90, 100, 200, 300, 400, 500, or more times lower than that on PDE4, the agent can be considered as a selective PDE4 inhibitor. The inhibitory activity of an agent on PDE4 can be tested by a well-known determination method in the art, for example, using a commercially available PDE4 kit. For example, a TR-FRET-based phosphodiesterase test kit from “Molecular Devices” is used to test whether an agent is a direct inhibitor of PDE4 enzyme.

In certain embodiments, the PDE4 inhibitor of the present application has a chemical structure shown below:

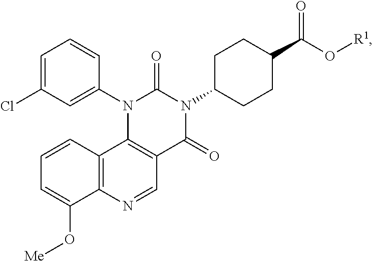

wherein R¹ is hydrogen or a (C₁-C₆) alkyl group optionally substituted with halo (e.g. fluoro, chloro, bromo, or iodo) or cyano (—CN) once, twice, or three times. In certain embodiments, R¹ is hydrogen. In certain embodiments, R¹ is methyl, ethyl, propyl, butyl (e.g., t-butyl), pentyl, or hexyl.

In another aspect, the present application provides use of a compound of Formula (I) or a pharmaceutically acceptable salt thereof in the manufacture of a medicament for reducing uric acid level in an individual:

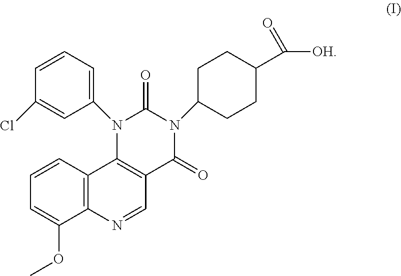 [SMILES: COc1cccc2c1ncc1c(=O)n(C3CCC(C(=O)O)CC3)c(=O)n(-c3cccc(Cl)c3)c12]

In another aspect, the present application provides use of a compound of Formula (I) or a pharmaceutically acceptable salt thereof in the manufacture of a medicament for preventing an increased uric acid level in an individual. In another aspect, the present application provides use of a compound of Formula (I) or a pharmaceutically acceptable salt thereof in the manufacture of a medicament for treating, preventing, or alleviating a disease, disorder, or condition associated with an increased uric acid level in an individual.

In the present application, the compound of Formula (I) refers to a compound with the following structure:

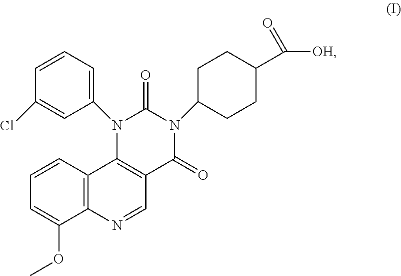 [SMILES: COc1cccc2c1ncc1c(=O)n(C3CCC(C(=O)O)CC3)c(=O)n(-c3cccc(Cl)c3)c12]

wherein the chemical name thereof is 4-[1-(3-chlorophenyl)-7-methoxy-2,4-dioxo-pyrimido[5,4-c]quinolin-3-yl]cyclohexanecarboxylic acid. In the present application, “the compound of Formula (I)” is also referred to as “Compound I”.

Compound I is an enantiomer, having a chemical structure of Formula (Ia) and Formula (Ib) respectively:

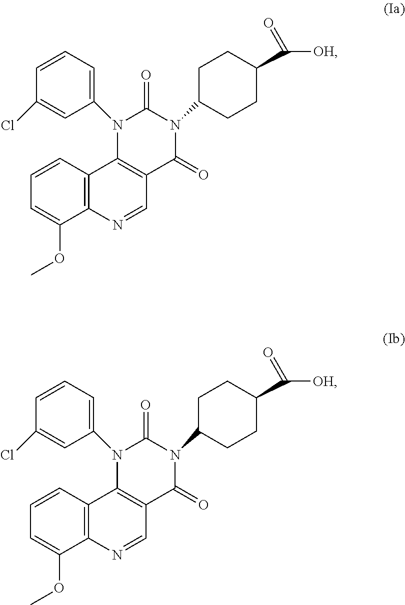

wherein the chemical names thereof are trans-4-[1-(3-chlorophenyl)-7-methoxy-2,4-dioxo-pyrimido[5,4-c]quinolin-3-yl]cyclohexanecarboxylic acid (also referred to as “Compound Ia” in the present application), cis-4-[1-(3-chlorophenyl)-7-methoxy-2,4-dioxo-pyrimido[5,4-c]quinolin-3-yl]cyclohexanecarboxylic acid (also referred to as “Compound Ib” in the present application). In certain embodiments, Compound I is Compound Ia. In certain embodiments, Compound I is Compound Ib. In certain embodiments, Compound I is a racemate containing compound Ia and compound Ib.

In the present application, “racemate” refers to a mixture of two or more optical isomers with different optical rotation properties. For example, a compound with one chiral center can have two optical isomers, one has a chiral center of R configuration, and the other has a chiral center of S configuration. For this compound, the racemate includes both the optical isomer of R configuration and the optical isomer of S configuration. In the racemate described in the present application, different optical isomers can exist as equal molar amounts (i.e., optical rotation cancellation), or as unequal molar amounts.

In the present application, unless otherwise indicated, the structure described in the present application also means to include a compound that differs only in the presence of one or more isotope-enriched atoms. For example, the compound with a structure of Formula (I), wherein hydrogen atom(s) is/are replaced by deuterium or tritium, or carbon atom(s) is/are replaced by carbon enriched in ¹³C or ¹⁴C, are within the scope of the present invention.

In the present application, the “pharmaceutically acceptable salt” refers to a salt prepared from pharmaceutically acceptable inorganic or organic acid and base. For example, Compound I can react with a number of inorganic acids and organic acids to form pharmaceutically acceptable acid addition salts, such as, but not limited to, hydrochloric, hydrobromic, hydroiodic, sulfuric, phosphoric, formic, acetic, propionic, citric, tartaric, and benzoic acids. Pharmaceutically acceptable base addition salts can be prepared from inorganic and organic bases. Salts derived from inorganic bases, include by ways of example, sodium, potassium, lithium, ammonium, calcium, and magnesium salts. Salts derived from organic bases include, but are not limited to, salts of primary, secondary and tertiary amines. Specific examples of such amines include, by way of example, isopropylamine, trimethyl amine, diethyl amine, tri(isopropyl) amine, tri(n-propyl) amine, ethanolamine, 2-dimethylaminoethanol, tromethamine, lysine, arginine, histidine, caffeine, procaine, choline, betaine, and ethylenediamine. Such pharmaceutically acceptable salts and common methodology for preparing them are well known in the art. See, for example, P. Stahl et al., HANDBOOK OF PHARMACEUTICAL SALTS: PROPERTIES, SELECTION AND USE (VCHA/Wiley-VCH, 2002); S. M. Berge, et al., “Pharmaceutical Salts”, Journal of Pharmaceutical Sciences, Vol. 66, No. 1, January 1977.

In certain embodiments, the pharmaceutically acceptable salt of Compound I described in the present application is an acid salt of Compound I. In certain embodiments, the pharmaceutically acceptable salt of Compound I described in the present application is a hydrochloric salt of Compound I.

In the present application, the “individual” includes human and non-human animals. Non-human animals include all vertebrates, for example, mammals and non-mammals (for example, non-human primates, mice, rats, cats, rabbits, sheep, dogs, cows, chickens, amphibians and reptiles). Unless otherwise specified, the term “patient”, “subject”, or “individual” can be used interchangeably in the present application.

In an embodiment, the individual described in the present application is a mammal. In another embodiment, the individual described in the present application is a human. In another embodiment, the individual described in the present application is a human having one or more symptom characteristics of a disorder in need of treatment. In certain embodiments, the individual has increased uric acid levels.

In the present application, “increased uric acid level” means that the uric acid level in a biological sample from an individual is found to be higher than a corresponding reference level thereof by comparing it with the corresponding reference level thereof. It is to be understood that “comparison” as used herein refers to the comparison of corresponding parameters or values, for example, the comparison of an absolute quantity with an absolute reference quantity, the comparison of a concentration with a reference concentration, or the comparison of an intensity signal obtained from a detected sample with an intensity signal of the same type of a reference sample. The comparisons can be made manually or with computer assistance. For computer-assisted comparison, the value of the determined quantity can be compared with the value of an appropriate reference stored in a database by a computer program. The result of comparison can be further evaluated by the computer program, and the desired evaluation can be automatically provided in an appropriate output format. By comparing the uric acid level in a biological sample from an individual with a corresponding reference level thereof, an individual suffering from a disease, disorder, or condition associated with an increased uric acid level can be identified, and the response of an individual to the treatment can also be monitored in the individual treated with a PDE4 inhibitor (for example, Compound I or a pharmaceutically acceptable salt thereof).

In the present application, the term “reference level” refers to a threshold level for including or excluding an individual in or from a criterion of increased uric acid level, or a threshold level used to monitor the response of the individual to treatment in individuals receiving a PDE4 inhibitor (for example, Compound I or a pharmaceutically acceptable salt thereof).

For example, in a method for reducing the uric acid level in an individual, if the uric acid level in the test sample is higher than a corresponding reference level thereof, the individual can be considered as having an increased uric acid level. In some embodiments, the uric acid level in the test sample is 1, 1.1, 1.2, 1.3, 1.4, 1.5, 1.6, 1.7, 1.8, 1.9, 2, 3, 4, 5, 6, 7, 8, 9, 10 or more times of the corresponding reference level thereof.

For another example, in the method for preventing an increased uric acid level in an individual, if the uric acid level in the test sample is lower than the corresponding uric acid level before treatment with the PDE4 inhibitor (for example, Compound I or a pharmaceutically acceptable salt thereof) or does not further increase according to the original progress, the PDE4 inhibitor (for example, Compound I or a pharmaceutically acceptable salt thereof) is considered to be able to preventing an increased uric acid level in the individual. In some embodiments, the uric acid level in a biological sample obtained after treatment with the PDE4 inhibitor (for example, Compound I or a pharmaceutically acceptable salt thereof) is at least 10%, 20%, 30%, 40%, 50%, 60%, 70%, 80%, 90%, 95%, 99% or 100% lower than the corresponding uric acid level before treatment.

The reference level of uric acid level can be derived from one or more reference samples, wherein the reference level is obtained from an experiment conducted in parallel with the experiment for testing the target sample. Alternatively, the reference level can be obtained in a database that includes a set of data, standards or levels from one or more reference samples or disease reference samples. In some embodiments, such sets of data, standards or levels are standardized so that they can be used for comparison purposes with data from one or more samples. “Standardization” is a process of converting the original measurement data into data that can be directly compared with other standardized data. Standardization is used to overcome the analytical-method-specific errors caused by different factors in different analytical methods, such as different amounts of sample load, different binding efficiency, different detection sensitivity and other kinds of errors.

Those skilled in the art can select the reference level according to the desired sensitivity and specificity. Means for determining an appropriate reference level are known to those skilled in the art. For example, the reference level can be determined from data collected in clinical research. For example, the reference level of uric acid level is 400 μmol/L, 410 μmol/L, 420 μmol/L, 430 μmol/L, 440 μmol/L, 450 μmol/L, or higher. In certain embodiments, if the uric acid level (e.g., serum uric acid level) in an individual is higher than 420 μmol/L, the individual can be considered as having an increased uric acid level. In certain embodiments, if twice uric acid levels (e.g., serum uric acid level) in an individual on different days are higher than 420 μmol/L, the individual can be considered as having an increased uric acid level. In certain embodiments, if twice fasting serum uric acid levels in an individual on different days are higher than 420 μmol/L, the individual can be considered as having an increased uric acid level.

Those skilled in the art can also determine the uric acid level in a biological sample (e.g., serum) by conventional methods in the art, for example, voltammetry, chromatography, capillary electrophoresis, isotope dilution mass spectrometry, and surface plasmon resonance, etc.

In the present application, the term “biological sample” or “sample” refers to a biological composition obtained from or derived from a target subject, which contains cells and/or other molecular entities to be characterized and/or identified, based on, for example, physical, biochemical, chemical and/or physiological characteristics. The biological samples include, but are not limited to, cells, tissues, organs and/or biological fluids of a subject obtained by any method known to those skilled in the art. In some embodiments, the biological sample is a fluid sample. In some embodiments, the fluid sample is whole blood, plasma, serum, mucus (including nasal excrement and sputum), peritoneal fluid, pleural fluid, pleural effusion, saliva, urine, synovial fluid, cerebrospinal fluid (CSF), pleural puncture fluid, abdominal fluid, ascites, or pericardial effusion. In some embodiments, the biological sample is a tissue or cell obtained from the heart, liver, spleen, lung, kidney, skin, or blood vessels of a subject.

In certain embodiments, the uric acid level described in the present application is a uric acid level in a fluid sample from an individual. In certain embodiments, the uric acid level described in the present application is a uric acid level in the blood from an individual. In certain embodiments, the uric acid level described in the present application is a uric acid level in the serum from an individual.

In the present application, the “treatment”, “therapy”, “prevention” or “alleviation” of a certain disease, disorder, or condition comprising preventing or alleviating a certain disease, disorder, or condition, reducing the speed of occurrence or development of a certain disease, disorder, or condition, reducing the risk of developing a certain disease, disorder, or condition, preventing or delaying the development of symptoms related to a certain disease, disorder, or condition, reducing or terminating symptoms related to a certain disease, disorder, or condition, generating a complete or partial reversal of a certain disease, disorder, or condition, curing a certain disease, disorder, or condition, or a combination thereof.

In certain embodiments, the disease, disorder, or condition associated with an increased uric acid level in the present application is selected from the group consisting of: gout, hyperuricemia, high uric acid level that does not reach the level usually diagnosed as hyperuricemia, renal dysfunction, urolithiasis, renal calculus, renal failure, diabetes, diabetes associated conditions, insulin resistance, metabolic syndrome, hypertension, hypothyroidism, hyperparathyroidism, obesity, inflammation, muscle spasm, local swelling, inflammation, joint pain, myocardial infarction, arthritis, tumor lysis syndrome, cognitive impairment, lead poisoning, psoriasis, sarcoidosis, non-alcoholic fatty liver disease, non-alcoholic fatty hepatitis, cardiovascular diseases, atherosclerosis, and transplants of blood, bone marrow or solid organs.

In certain embodiments, the disease, disorder, or condition associated with an increased uric acid level mentioned in the present application is gout. In certain embodiments, the disease, disorder, or condition associated with an increased uric acid level mentioned in the present application is hyperuricemia. In certain embodiments, the hyperuricemia is primary hyperuricemia or secondary hyperuricemia. In certain embodiments, the secondary hyperuricemia includes hyperuricemia related to drugs or hyperuricemia related to various medical conditions.

### 2. Medicament

In another aspect, the present application provides a medicament comprising a PDE4 inhibitor (for example, Compound I or a pharmaceutically acceptable salt thereof) and a pharmaceutically acceptable carrier.

In the present application, the “pharmaceutically acceptable carrier” refers to a carrier that is generally chemically and/or physically compatible with other ingredients in the formulation, and physiologically compatible with its recipient.

The pharmaceutically acceptable carriers used in the pharmaceutical composition disclosed in the present invention may include, for example, a pharmaceutically acceptable liquid, a gel or a solid carrier, an aqueous solvent, a non-aqueous solvent, an antimicrobial substance, an isotonic substance, a buffer, an antioxidant, an anesthetic, a suspending agent/dispersant, a chelating agent, a diluent, an adjuvant, an auxiliary material or a non-toxic auxiliary substance, and other components known in the art or various combinations thereof.

Suitable components may include, for example, an antioxidant, a filler, a binder, a disintegrant, a buffer, a preservative, a lubricant, a flavoring agent, a thickening agent, a coloring agent, an emulsifier, or a stabilizer such as sugar and cyclodextrin. Suitable antioxidants may include, for example, methionine, ascorbic acid, EDTA, sodium thiosulfate, platinum, hydrogen peroxidase, citric acid, cysteine, mercaptoglycerol, mercaptoacetic acid, mercaptosorbitol, butyl methyl anisole, butylated hydroxytoluene and/or propyl gallate. As disclosed in the present invention, the pharmaceutical composition comprising a PDE4 inhibitor (for example, Compound I or a pharmaceutically acceptable salt thereof) further comprises one or more antioxidants such as methionine, to reduce the oxidation of the PDE4 inhibitor (for example, Compound I or a pharmaceutically acceptable salt thereof). The present invention further provides various methods for preventing the oxidation, prolonging the shelf life and/or improving the activity of the PDE4 inhibitor (for example, Compound I or a pharmaceutically acceptable salt thereof), for example, by mixing the PDE4 inhibitor (for example, Compound I or a pharmaceutically acceptable salt thereof) provided in the present invention with one or more antioxidants (for example, methionine).

The pharmaceutical composition in the present application can be formulated into a unit dose form containing a predetermined amount of active ingredient per unit dose. As a non-limiting example, such a predetermined amount or unit dose may contain 0.5 mg to 1 g (for example, no more than 5 mg, 10 mg, 15 mg, 20 mg, 25 mg, 30 mg, 35 mg, 40 mg, 45 mg, 50 mg, 60 mg, 70 mg, 80 mg, 90 mg, or 100 mg) of a PDE4 inhibitor (for example, Compound I or a pharmaceutically acceptable salt thereof), depending on the condition being treated, the route of administration, and the age, weight, and condition of the patient. A typical unit dosage formulation is that containing a daily dose or sub-dose or an appropriate fraction thereof, of an active ingredient. Such a pharmaceutical composition may be prepared by any of the methods known in the pharmacy art.

In an embodiment, a unit dosage form of a pharmaceutical composition may comprise a PDE4 inhibitor (for example, Compound I or a pharmaceutically acceptable salt thereof), in an amount greater than 1 mg of the PDE4 inhibitor (for example, Compound I or a pharmaceutically acceptable salt thereof). In another embodiment, the pharmaceutical composition may comprise a PDE4 inhibitor (for example, Compound I or a pharmaceutically acceptable salt thereof), in an amount between 1 and 100 mg, or between 1 and 50 mg, or between 10 and 50 mg, or between 30 and 50 mg. In other embodiments, the pharmaceutical composition may comprise a PDE4 inhibitor (for example, Compound I or a pharmaceutically acceptable salt thereof), in an amount between 1 and 20 mg, or between 5 and 15 mg, or between 10 and 20 mg, or between 20 and 30 mg.

The pharmaceutical composition may be adapted for administration by any appropriate route, for example, oral (including buccal or sublingual), rectal, nasal, topical (including buccal, sublingual or transdermal), vaginal or parenteral (including subcutaneous, intramuscular, intraperitoneal, intravenous or intradermal) route. Such pharmaceutical compositions may be prepared by any method known in the art of pharmacy, for example, by bringing into association the active ingredient with carrier(s) or excipient(s). By way of example, and not meant to limit the invention, with regard to certain conditions and disorders for which the compound of the present invention are believed useful, certain routes will be preferable to others.

Pharmaceutical compositions suitable for oral administration may be presented as discrete units such as capsules or tablets; powders or granules; solutions or suspensions, each with aqueous or non-aqueous liquid; edible foams or whips; or oil-in-water liquid emulsions or water-in-oil liquid emulsions. For example, for oral administration in the form of a tablet or capsule, the active drug component can be combined with an oral, non-toxic pharmaceutically acceptable inert carrier such as ethanol, glycerol, and water, and the like. Generally, powders are prepared by comminuting the compound to a suitable fine size and mixing it with an appropriate pharmaceutical carrier such as an edible carbohydrate, as, for example, starch or mannitol. Flavorings, preservatives, dispersing agents and coloring agents can also be present.

Capsules are made by preparing a powder, liquid or suspension mixture and encapsulating it with gelatin or some other appropriate shell material. Glidants and lubricants such as colloidal silica, talc, magnesium stearate, calcium stearate, or solid polyethylene glycol can be added to the mixture before encapsulation. A disintegrant or a solubilizing agent, such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like.

Lubricants useful in these dosage forms include, for example, sodium oleate, sodium stearate, magnesium stearate, sodium benzoate, sodium acetate, sodium chloride, and the like. Disintegrators includes, but is not limited to, starch, methyl cellulose, agar, bentonite, xanthan gum, and the like.

Tablets are formulated, for example, by preparing a powder mixture, granulating or slugging, adding a lubricant and a disintegrant, and pressing into tablets. A powder mixture may be prepared by mixing the compound, suitably comminuted, with a diluent or base as described above. Optional ingredients include binders such as carboxymethylcellulose, alginates, gelatins, or polyvinyl pyrrolidone, solution retardants such as paraffin, resorption accelerators such as a quaternary salt, and/or absorption agents such as bentonite, kaolin or dicalcium phosphate. The powder mixture can be wet-granulated with a binder, such as syrup, starch paste, acadia mucilage or solutions of cellulosic or polymeric materials, and forcing through a screen. As an alternative to granulating, the powder mixture can be run through the tablet machine, and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or a polymeric material, and a polishing coating of wax can be provided. Dyestuffs can be added to these coatings to distinguish different unit doses.

Where appropriate, unit dosage formulations for oral administration can be microencapsulated. The formulation can also be prepared to prolonged or sustain the release as for example by coating or embedding particulate materials in polymers, wax, and the like.

Oral liquids such as solutions, syrups, and elixirs can be prepared in dosage unit form so that a administrated quantity contains a predetermined amount of a compound. Syrups can be prepared, for example, by dissolving the compound in a suitably flavored aqueous solution, while elixirs are prepared through the use of a non-toxic alcohol vehicle. Suspensions can be formulated generally by dispersing the compound in a nontoxic vehicle. Solubilizers and emulsifiers such as ethoxylated isostearyl alcohols and polyoxy ethylene sorbitol ethers, preservatives flavor additives such as peppermint oil, or natural sweeteners, saccharin, or other artificial sweeteners, and the like can also be added.

Suitable packages for pharmaceutical solution formulations may be all approved containers intended for parenteral use, such as plastic and glass containers, ready-to-use syringes, and the like. In an embodiment, the container is a sealed glass container, such as a vial or an ampoule. A hermetically glass vial is one example of a sealed glass container. According to an embodiment of the present invention, there is provided, in a sealed glass container, a sterile, injectable solution comprising Compound I or a pharmaceutically acceptable salt thereof in a physiologically acceptable solvent, and which has an appropriate pH for stability. Acid salt of the compounds of the present invention may be more soluble in aqueous solutions than their free base counter parts, but when the acid salts are added to aqueous solutions the pH of the solution may be too low to be suitable for administration. Thus, solution formulations having a pH above 4.5 may be combined prior to administration with a diluent solution of pH greater than 7 such that the pH of the combined formulation administrated is pH 4.5 or higher. In one embodiment, the diluent solution comprises a pharmaceutically acceptable base such as sodium hydroxide and the pH of the combined formulation administrated is between pH 5.0 and 7.0. One or more additional components such as a co-solubilizing agents, tonicity adjustment agents, stabilizing agents and preservatives, for instance of the kind previously specified, may be added to the solution prior to passing the solution through the sterilizing filter.

Pharmaceutical compositions adapted for topical administration may be formulated into ointments, creams, suspensions, lotions, powders, solutions, pastes, gels, sprays, aerosols or oil.

Pharmaceutical compositions adapted for topical administration in the mouth include lozenges, pastilles, and mouthwashes.

Pharmaceutical compositions adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered dose pressurized aerosols, nebulizers, or insufflators.

Further, the composition of the present invention may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example. Pharmaceutical compositions adapted for rectal administration may be presented as suppositories or as enemas.

Pharmaceutical compositions adapted for parenteral administration include aqueous and non-aqueous sterile injections which may contain anti-oxidants, buffers, bacteriostats and solutes that render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules, and tablets.

In addition to the ingredients particularly mentioned above, the formulations of the present invention may include other agents conventional in the art having regard to the type of formulation in question. For example, formulations suitable for oral administration may include flavoring or coloring agents.

In another aspect, the pharmaceutical composition of the present application further comprises one or more additional uric acid-reducing drugs. “Uric acid-reducing drugs” can be medicaments that reduce the uric acid synthesis or medicaments that promote uric acid excretion. “Uric acid-reducing drugs” can be uric acid-reducing drugs that have been approved for marketing by drug supervision and administration departments (for example, US Food and Drug Administration (USFDA), China National Medical Products Administration (NMPA), and European Medicines Administration (EMA)), or medicaments that have been proved to reduce the uric acid levels in preclinical research or clinical research, but have not been approved for marketing by drug supervision and administration departments. In certain embodiments, the additional uric acid-reducing drug is selected from the group consisting of: xanthine oxidase inhibitors, uricosuric agents, urate transporter-1 inhibitors, uricase, and inhibin. In certain embodiments, the additional uric acid-reducing drug is selected from the group consisting of: allopurinol, febuxostat, lesinurad, benzbromarone, roflumilast, apremilast, sodium bicarbonate, potassium sodium hydrogen citrate, potassium sodium citrate, non-steroidal anti-inflammatory drugs, glucocorticoids, and colchicine.

3. Treatment Method with PDE4 Inhibitor or Pharmaceutical Composition Thereof

In another aspect, the present application provides a method for reducing uric acid level, comprising the administration of a PDE4 inhibitor (for example, Compound I or a pharmaceutically acceptable salt thereof) to an individual in need. In another aspect, the present application further provides a method for preventing an increased uric acid level, comprising the administration of a PDE4 inhibitor (for example, Compound I or a pharmaceutically acceptable salt thereof) to an individual in need. In another aspect, the present application further provides a method for treating, preventing, or alleviating a disease, a disorder, or a condition associated with an increased uric acid level in an individual, comprising the administration of a PDE4 inhibitor (for example, Compound I or a pharmaceutically acceptable salt thereof) to an individual in need.

In certain embodiments, the disease, disorder, or condition associated with an increased uric acid level in the present application is selected from the group consisting of: gout, hyperuricemia, high uric acid level that does not reach the level usually diagnosed as hyperuricemia, renal dysfunction, urolithiasis, renal calculus, renal failure, diabetes, diabetes associated conditions, insulin resistance, metabolic syndrome, hypertension, hypothyroidism, hyperparathyroidism, obesity, inflammation, muscle spasm, local swelling, inflammation, joint pain, myocardial infarction, arthritis, tumor lysis syndrome, cognitive impairment, lead poisoning, psoriasis, sarcoidosis, non-alcoholic fatty liver disease, non-alcoholic fatty hepatitis, cardiovascular diseases, atherosclerosis, and transplants of blood, bone marrow or solid organs.

In certain embodiments, the disease, disorder, or condition associated with an increased uric acid level described in the present application is gout. In certain embodiments, the disease, disorder, or condition associated with an increased uric acid level described in the present application is hyperuricemia. In certain embodiments, the hyperuricemia is primary hyperuricemia or secondary hyperuricemia. In certain embodiments, the secondary hyperuricemia comprises hyperuricemia related to drugs or hyperuricemia related to various medical conditions.

In certain embodiments, the treatment method described in the present application includes administrating a PDE4 inhibitor (for example, Compound I) or a pharmaceutically acceptable salt thereof to an individual at a dosage between 6 mg and 40 mg, for example, 6 mg, 6.5 mg, 7 mg, 7.5 mg, 8 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 35 mg, 40 mg, and the like.

The dosage and/or the frequency of administration per day (for example, once a day, twice a day, three times a day, etc.) or per period (once a week, twice a week, three times a week, etc.) may be individualized by the clinician based on the specific clinical condition of the individual being treated. Thus, it will be understood that the specific dosage level and frequency of administration for any particular individual may depend upon a variety of factors such as, but not limited to, the age, body weight, general health, sex, diet, time of administration, route of administration, rate of excretion, drug combination and the severity of the particular disease undergoing therapy.

In certain embodiments, the PDE4 inhibitor (for example, Compound I or a pharmaceutically acceptable salt thereof) of the present application can also be administered in combination with an additional uric acid-reducing drug. In certain embodiments, the additional uric acid-reducing drug is selected from the group consisting of: xanthine oxidase inhibitors, uricosuric agents, urate transporter-1 inhibitors, uricase, and inhibin. In certain embodiments, the additional uric acid-reducing drug is selected from the group consisting of: allopurinol, febuxostat, lesinurad, benzbromarone, roflumilast, apremilast, sodium bicarbonate, potassium sodium hydrogen citrate, potassium sodium citrate, non-steroidal anti-inflammatory drugs, glucocorticoids, and colchicine.

When the PDE4 inhibitor (for example, Compound I or a pharmaceutically acceptable salt thereof) of the present application is administered in combination with an additional uric acid-reducing drug, such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of the PDE4 inhibitor (for example, Compound I or a pharmaceutically acceptable salt thereof) of the present application with the additional uric acid-reducing drug may be in combination by administration concomitantly in: (1) a unitary composition including both compounds, or (2) separate pharmaceutical compositions each including one of the compounds. Alternatively, the combination may be administered separately in an sequential manner wherein one treatment agent is administered first and the other second or vice versa. Such sequential administration may be at close in time or remote in time.

### EXAMPLES

### Example 1. Synthesis of Compound and Test of PDE4 Inhibiting Activity Thereof

For the synthesis method of Compound I and the test of PDE4 inhibiting activity, please refer to the corresponding description in the Examples of Chinese Patent No. CN111808098B (paragraph [0082] on page 10 to paragraph [0114] on page 14 of the specification).

### Example 2. Clinical Trial of Compound I

### 2.1 Design of Clinical Trial Scheme

| | |
|---|---|
| Drug name | Compound I |
| Research topic | A randomized, double-blind, placebo-controlled, dose-increasing phase |
| | I clinical study to evaluate the safety, tolerance, pharmacokinetics and |
| | pharmacodynamics of Compound I in healthy subjects |
| Research design | Single-dose and multiple-dose, randomized, double-blind, placebo- |
| | controlled, dose-increasing, single-center phase I clinical trial |
| Research object | Healthy subjects |
| Sample size | Expected 84 healthy subjects, male and female |
| | Single dose of about 48 cases: expected 6 mg, 10 mg, 20 mg, 40 mg, or |
| | other doses, 12 subjects in each group, Compound I to placebo ratio of |
| | 3:1 (9 cases:3 cases), 6 mg and 10 mg can be carried out simultaneously. |
| | The dosage group and the total number of subjects can be adjusted |
| | according to the latest data obtained. |
| | Multi-dose of about 36 cases: expected 10 mg, 20 mg, 40 mg, or other |
| | doses, 12 subjects in each group, Compound I to placebo ratio of 3:1 (9 |
| | cases:3 cases). The dosage group and the total number of subjects can |
| | be adjusted according to the latest data obtained. The multi-dose and |
| | single dose can be carried out simultaneously, and the subjects in each |
| | multi-dose group are included separately after the first subject in the |
| | same dose group included. |
| Test drug | Test drug Compound I or placebo capsules, Specification: 1 mg and 5 |
| | mg |
| Research | Main objective: |
| objective | To evaluate the safety and tolerability of Compound I in |
| | healthy subjects. |
| | Secondary objective: |
| | To establish the pharmacokinetic characteristics of |
| | Compound I in healthy subjects. |
| | Through the pharmacokinetic data of Compound I in |
| | Caucasian healthy subjects, to indirectly compare the |
| | pharmacokinetic characteristics of Compound I in Chinese |
| | population. |
| Criteria of | Subjects who meet all the following criteria of inclusion will be included |
| inclusion | in this study: |
| | 1. Sign the informed consent, and voluntarily participate in the study; |
| | 2. Male or female aged 18-45 (including 18 years and 45 years) at the |
| | time of screening; |
| | 3. Male or female who agrees to accept effective contraception from |
| | the screening period to 90 days after the last dose in the study; |
| | agree to take at least one or more effective contraceptive measures; |
| | 4. During the screening period and the baseline period, the body |
| | weight of male is not less than 50 Kg, the body weight of female is |
| | not less than 45 Kg, the body mass index (BMI) is in the range of |
| | 19-24 kg/m² (including 19 and 24), [BMI = weight (kg)/height² |
| | (m²)]; |
| Criteria of | Subjects who meet any of the following criteria of exclusion will be |
| exclusion | excluded from this study: |
| | 1. subjects having a specific allergic history or allergic constitution to |
| | drugs, food, and pollen, or subjects that are allergic to PDE4 |
| | inhibitors or similar drugs; |
| | 2. subjects having had diseases of neuropsychiatric system, |
| | respiratory system, cardiovascular system, digestive system, blood |
| | lymphatic system, liver and kidney system, endocrine system, |
| | musculoskeletal system, or other diseases, and the researcher |
| | determines that such past medical history may have an impact on |
| | the drug metabolism or safety; |
| | 3. screening or baseline shows abnormal results with clinical |
| | significance of vital signs, physical examination, laboratory |
| | examination, electrocardiogram, and the like; |
| | 4. subjects having a history of malignant tumor during the past 5 |
| | years; |
| | 5. subjects having abnormal chest X-ray or abdominal B-ultrasound |
| | with clinical significance; |
| | 6. subjects who are positive for hepatitis B surface antigen, hepatitis |
| | C virus antibody, human immunodeficiency virus antibody, or |
| | syphilis antibody; |
| | 7. subjects having a history of drug dependence or drug abuse, or |
| | positive in urine screening of drug abuse; |
| | 8. smokers (5 or more cigarettes a day); |
| | 9. alcoholics (drinking more than 14 units per week, each unit is |
| | equivalent to 360 mL of beer or 150 mL of wine or 45 mL of liquor |
| | with 40% alcohol content); |
| | 10. subjects having participated in any clinical trials of drugs or |
| | medical devices within 3 months before screening; |
| | 11. subjects who are menstruating, pregnant or breastfeeding females, |
| | or tested positive for pregnancy at the time of screening or baseline; |
| | 12. subjects having taken any prescription drugs and Chinese herbal |
| | medicines within 4 weeks before enrollment, or having taken over- |
| | the-counter drugs or dietary supplements (including vitamins, |
| | calcium supplements, etc.) within 2 weeks before enrollment; |
| | 13. subjects having blood donation or blood loss of ≥400 mL or |
| | received blood transfusion within 3 months before screening; or |
| | subjects have blood donation or blood loss of ≥200 mL within 1 |
| | month before screening; |
| | 14. subjects having a history of serious infection, trauma or major |
| | surgery within 4 weeks before screening; |
| | 15. subjects who cannot tolerate venous puncture blood collection or |
| | have a history of needle fainting and blood fainting; |
| | 16. subjects who cannot guarantee to abstain from ethanol, cigarette, |
| | food and drinks containing xanthine or caffeine (including |
| | chocolate, tea, coffee, cola, etc.) and to avoid strenuous exercise |
| | from 48 hours before administration to the end of the trial period; |
| | 17. subjects who cannot guarantee to abstain from dragon fruit, |
| | grapefruit, mango, carambola, and fruit juice containing the above |
| | fruits, and spicy food from 7 days (i.e. 1 week) before |
| | administration and during the whole trial period; |
| | 18. subjects who cannot swallow the test drug, or have absorption |
| | disorder caused by gastrointestinal diseases; |
| | 19. subjects who cannot participate in this trial according to the |
| | researcher's judgment. |
| Criteria of | Subject may withdraw from the study at any time for any reason, without |
| withdrawal | any punishment or influence on further disease treatment. If needed by |
| | the clinical condition of the subject, the researcher may decide the |
| | subject to withdraw from the study at any time. Criteria for subjects to |
| | withdraw from the study: |
| | 1) the subject requests to withdraw from the study; |
| | 2) the researcher, the sponsor, and the institutional committee of the |
| | ethics committee requests the subject to withdraw from the study |
| | for safety, supervision or other reasons, such as the subjects' |
| | apparent non-compliance with the study procedures and/or |
| | treatment; |
| | 3) death; |
| | 4) loss to visit; or |
| | 5) pregnancy. |
| Termination of | The trial will be terminated if the following cases happen: |
| trial | 1) large-scale and unexpected serious adverse reactions occur in the |
| | clinical trial of drugs, or when there is evidence that the drug used |
| | in the clinical trial has serious quality problems, the sponsor needs |
| | to stop the clinical trial; |
| | 2) cases of serious violation of clinical trial protocol, GCP, or relevant |
| | laws and regulations occur, so that the trial needs to be terminated; |
| | 3) on the premise of fully protecting the rights and safety of the |
| | subjects, the sponsor requires to terminate (such as for financial |
| | reasons and management reasons); or |
| | 4) National Medical Products Administration (or equivalent agencies) |
| | or the Ethics Committee orders the termination of the trial for some |
| | reason. |
| Administration | In each dose group, the subjects will be randomly assigned to the |
| regimen | Compound I group or the placebo group at a ratio of 3:1 (i.e., 9 cases of |
| | Compound I; and 3 cases of placebo), the test drug is orally |
| | administrated as follows: |
| | Single dose: 1 day, once a day, orally taken 30 min after breakfast. |
| | Multiple dose: 7 consecutive days, once a day, orally taken 30 min after |
| | breakfast |
| | During the study period, it is recommended to take the drug at roughly |
| | the same time every day (for example, about 8:00 am to 9:00 am). |
| | In case of vomiting, the subject is not recommended to take the test drug |
| | again. |
| Evaluation | Main evaluation indexes |
| indexes | Safety and tolerance: |
| | Incidence and severity of various adverse events, including |
| | subjects' physical examination, routine blood test, blood biochemistry, |
| | urine flow sediment detection, coagulation function, vital signs (pulse, |
| | respiration, blood pressure, and body temperature), electrocardiogram |
| | and the like. |
| | Routine blood test: hemoglobin, red blood cell count, white |
| | blood cell count, white blood cell classification (neutrophils, |
| | monocytes, eosinophils, basophils, and lymphocytes), and |
| | platelets. |
| | Coagulation function test: prothrombin time (PT), activated |
| | partial thromboplastin time (aPTT), fibrinogen and |
| | international normalized ratio INR. |
| | Blood biochemical test: sodium, potassium, chloride, |
| | calcium, phosphorus, magnesium, blood urea nitrogen |
| | (BUN) or urea, creatinine, uric acid, alkaline phosphatase, |
| | total protein, albumin, glucose, total cholesterol, total |
| | triglyceride, high-density lipoprotein, low-density |
| | lipoprotein and liver function test (including GGT, AST, |
| | ALT, lactate dehydrogenase (LDH), total bilirubin, direct |
| | bilirubin, and indirect bilirubin). |
| | Urine flow sediment detection: proteins, glucose, pH, |
| | ketones, specific gravity, bilirubin, and urobilinogen; and |
| | microscopic examination of white blood cells and red blood |
| | cells. |
| | Pharmacokinetics (PK): |
| | Plasma Compound I concentration and pharmacokinetic related |
| | parameters. |
| | Time to peak (Tₘₐₓ), peak concentration (Cₘₐₓ), area under drug |
| | concentration-time curve from time 0 to the last time t that the |
| | concentration can be accurately measured (AUC₀₋ₜ), area under drug |
| | concentration-time curve from time 0 to infinite time (AUC_{0-inf}), |
| | elimination rate constant (λ_{z}), elimination half-life (t_{1/2}), apparent |
| | clearance rate (CL/F), apparent volume of distribution (V_{z}/F), |
| | extrapolated percentages of AUC (% AUCₑₓ), and the like. |
| | Time to steady-state peak (T_{ss, max}), steady-state peak concentration |
| | (C_{ss, max}), steady-state valley concentration (C_{ss, min}), steady-state average |
| | concentration (C_{ss, avg}), area under plasma concentration-time curve from |
| | the last administration to the time t when the concentration can be |
| | accurately measured (AUC₀₋ₜ), area under drug concentration-time curve |
| | from the last administration to infinite time (AUC_{0-inf}), elimination rate |
| | constant (λ_{z}), elimination half-life (t_{1/2}), extrapolated percentages of |
| | AUC (% AUCₑₓ), and the like. |
| Statical method | Statistical description method are mainly used for test results. |
| | Quantitative data of mean, standard deviation, median, maximum, and |
| | minimum values are listed; and qualitative data of the frequency and |
| | relative frequency (relative ratio) are listed. |
| | Safety data: for adverse events, adverse events occurring or |
| | aggravated after administration will be summarized according to each |
| | dose group. If other safety data are consistent with the nature of |
| | quantitative data, then the baseline data, post-administration data, and |
| | post-administration changes will be summarized according to each visit |
| | and each dose group. For qualitative data, such as the normality and |
| | clinical significance of various tests, a transition table will be used to |
| | describe the changes from baseline to each visit after administration. PK |
| | data: The concentration of Compound I in plasma will be summarized |
| | according to each dose group and sampling time point; and the PK |
| | parameters will be summarized according to each dose group. This |
| | section will be reported additionally. |
| | The intention-to-treat (ITT) set is defined as all subjects assigned |
| | by randomized treatment according to the protocol. |
| | The safety set (SS) is defined as the subjects who have taken the |
| | test drug at least once and underwent safety assessment. SS population |
| | is the main population for safety evaluation in this study. |
| | Pharmacokinetic set (PKS) is defined as all subjects in the |
| | population having at least one measurable blood sample after |
| | administration. PKS population is the main population for |
| | pharmacokinetic evaluation in this study. |
| Progress of test | After the project is approved and initiated, the LSLV (last subject last |
| | visit, i.e., the study is completed) is completed in about 12 months. |

### 2.2 Clinical Trial Results

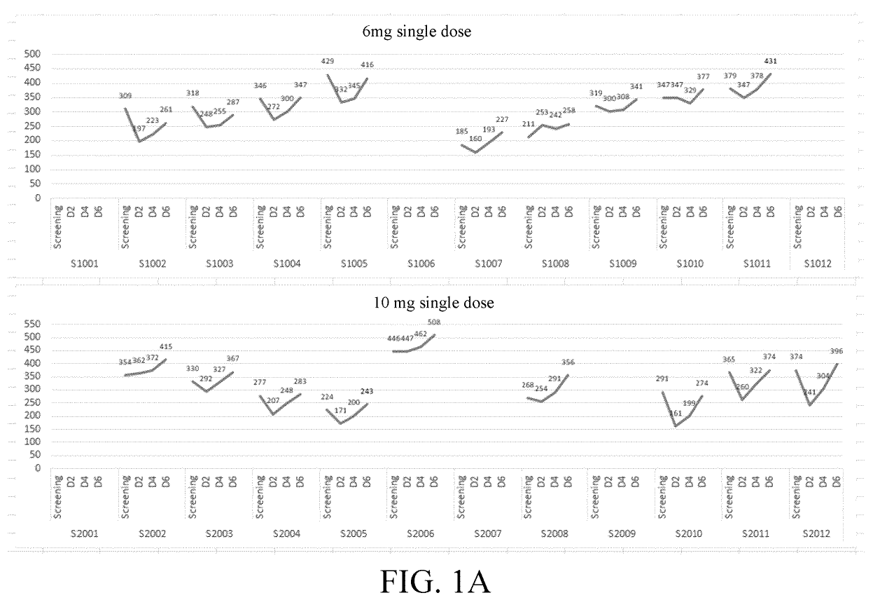

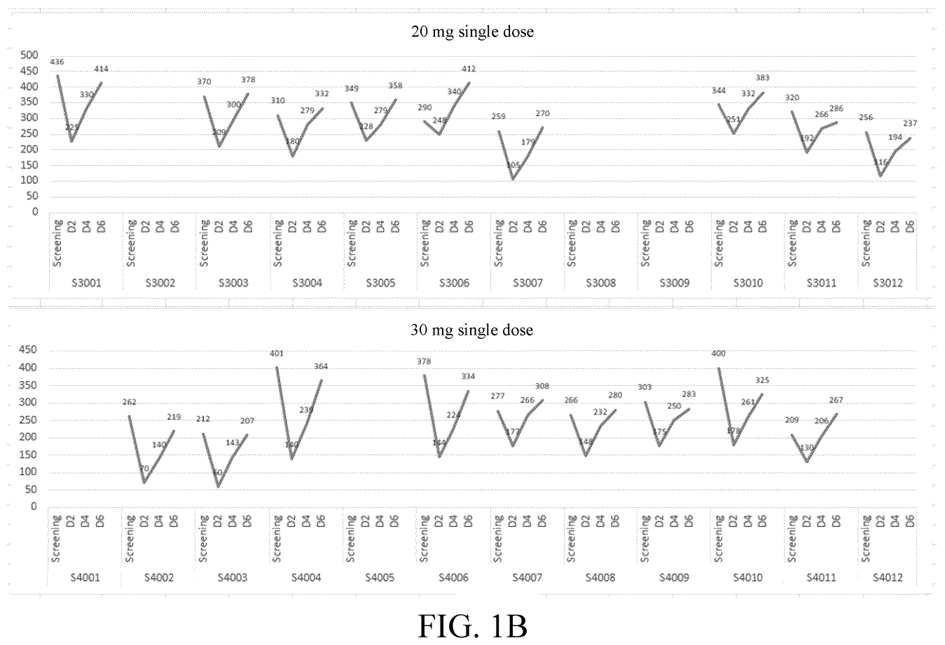

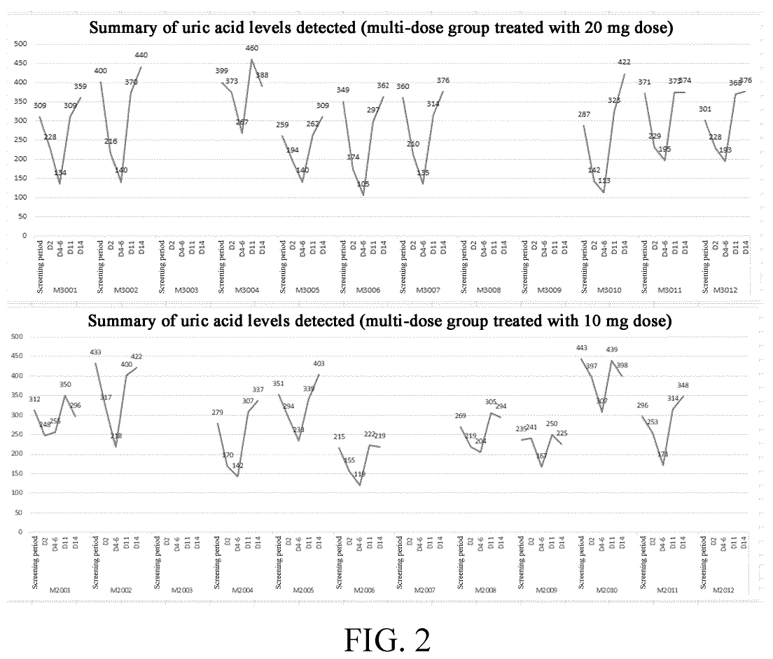

The changes of uric acid levels in subjects in single-dose and multi-dose groups of Compound I are shown in FIG. 1A (single dose of 6 mg and single dose of 10 mg), FIG. 1B (single dose of 20 mg and single dose of 30 mg), and FIG. 2 (multi-dose of 10 mg and multi-dose of 20 mg). The corresponding results are also shown in Tables 1.1, 1.2, 1.3, and 2.1, and tables 2.2, 2.3, and 2.4. Wherein Tables 1.1, 1.2, and 1.3 show the changes of uric acid levels in subjects in single-dose groups (6 mg, 10 mg, 20 mg, and 30 mg) before and after treatment with Compound I. Tables 2.1, 2.2, 2.3, and 2.4 show the changes of uric acid levels in subjects in multi-dose groups (10 mg and 20 mg) before and after treatment with Compound I. According to the relevant data, after administration in the single-dose groups: the uric acid level decreases obviously the next day, showing a certain dose dependence; and the detection on the following days (days 4 and 6) shows that the uric acid level in the treatment groups is gradually recovered. After administration in the multi-dose groups: the uric acid level decreases obviously the next day, showing a certain dose dependence; and the detection on the following days (days 4-6) shows that the uric acid level in the treatment group has a continuous decrease. These clinical trial results show that Compound I reduces the uric acid level in the subjects significantly (p<0.05), and has the potential to be a candidate drug for reducing the uric acid level, preventing an increased uric acid level, or treating, preventing, or alleviating a disease, disorder, or condition associated with an increased uric acid level in an individual.

TABLE 1.1
Changes of uric acid index before and after treatment with Compound I
Single-dose groups
Safety analysis set
Index: Uric acid (UA) (μmol/L)

| | | Compound I | Compound I | Compound I | Compound I | |
|---|---|---|---|---|---|---|
| Visit | Placebo | 6 mg | 10 mg | 20 mg | 30 mg | Total |
| Statistics | (N = 12) | (N = 9) | (N = 9) | (N = 9) | (N = 9) | (N = 48) |
| Baseline | | | | | | |
| Number of | 12 | 9 | 9 | 9 | 9 | 48 |
| subjects | | | | | | |
| Mean (standard | 358.6 | 315.9 | 335.2 | 326.0 | 300.9 | 329.3 |
| deviation) | (59.33) | (76.52) | (77.72) | (56.86) | (75.37) | (69.16) |
| Median | 344.5 | 319.0 | 330.0 | 320.0 | 277.0 | 330.5 |
| Minimum value, | 261, 455 | 185, 429 | 224, 446 | 256, 436 | 209, 401 | 185, 455 |
| maximum value | | | | | | |
| Day 2 | | | | | | |
| Number of | 12 | 9 | 9 | 9 | 9 | 48 |
| subjects | | | | | | |
| Mean (standard | 374.2 | 272.9 | 266.1 | 194.9 | 135.8 | 256.6 |
| deviation) | (52.67) | (65.89) | (91.51) | (53.27) | (43.81) | (103.66) |
| Median | 355.5 | 272.0 | 254.0 | 209.0 | 144.0 | 249.5 |
| Minimum value, | 292, 451 | 160, 347 | 161, 447 | 105, 251 | 60, 178 | 60, 451 |
| maximum value | | | | | | |
| Changes over | | | | | | |
| baseline on day 2 | | | | | | |
| Number of | 12 | 9 | 9 | 9 | 9 | 48 |
| subjects | | | | | | |
| Mean (standard | 15.6 | −43.0 | −69.1 | −131.1 | −165.1 | −72.7 |
| deviation) | (25.71) | (49.32) | (47.93) | (46.62) | (64.54) | (80.55) |
| Median | 19.0 | −32.0 | −70.0 | −130.0 | −152.0 | −72.0 |
| Minimum value, | −39, 68 | −112, 42 | −133, 1 | −211, −42 | −261, −79 | −261, 68 |
| maximum value | | | | | | |
| P value | 0.060 | 0.031* | 0.003* | <0.001* | <0.001* | |

Abbreviation: N = Number of subjects in safety analysis set.
P value is obtained from paired t test.
*P < 0.05.

TABLE 1.2
Changes of uric acid index before and after treatment with Compound I
Single-dose groups
Safety analysis set
Index: Uric acid (UA) (μmol/L)

| | | Compound I | Compound I | Compound I | Compound I | |
|---|---|---|---|---|---|---|
| Visit | Placebo | 6 mg | 10 mg | 20 mg | 30 mg | Total |
| Statistics | (N = 12) | (N = 9) | (N = 9) | (N = 9) | (N = 9) | (N = 48) |
| Day 4 | | | | | | |
| Number of | 12 | 9 | 9 | 9 | 9 | 48 |
| subjects | | | | | | |
| Mean (standard | 347.3 | 285.9 | 302.8 | 277.7 | 217.9 | 290.1 |
| deviation) | (50.03) | (61.23) | (83.35) | (58.04) | (47.04) | (72.49) |
| Median | 332.5 | 300.0 | 304.0 | 279.0 | 232.0 | 298.0 |
| Minimum value, | 285, 424 | 193, 378 | 199, 462 | 179, 340 | 140, 266 | 140, 462 |
| maximum value | | | | | | |
| Changes over | | | | | | |
| baseline on day 4 | | | | | | |
| Number of | 12 | 9 | 9 | 9 | 9 | 48 |
| subjects | | | | | | |
| Mean (standard | −11.3 | −30.0 | −32.4 | −48.3 | −83.0 | −39.1 |
| deviation) | (33.95) | (41.70) | (40.08) | (45.79) | (62.27) | (49.64) |
| Median | −9.5 | −18.0 | −29.0 | −62.0 | −69.0 | −32.0 |
| Minimum value, | −85, 46 | −86, 31 | −92, 23 | −106, 50 | −162, −3 | −162, 50 |
| maximum value | | | | | | |
| P value | 0.275 | 0.063 | 0.041* | 0.013* | 0.004* | |

Abbreviation: N = Number of subjects in safety analysis set.
P value is obtained from paired t test.
*P < 0.05.

TABLE 1.3
Changes of uric acid index before and after treatment with Compound I
Single-dose groups
Safety analysis set
Index: Uric acid (UA) (μmol/L)

| | | Compound I | Compound I | Compound I | Compound I | |
|---|---|---|---|---|---|---|
| Visit | Placebo | 6 mg | 10 mg | 20 mg | 30 mg | Total |
| Statistics | (N = 12) | (N = 9) | (N = 9) | (N = 9) | (N = 9) | (N = 48) |
| Last visit (day 6) | | | | | | |
| Number of | 12 | 9 | 9 | 9 | 9 | 48 |
| subjects | | | | | | |
| Mean (standard | 361.9 | 327.2 | 357.3 | 341.1 | 287.4 | 336.7 |
| deviation) | (44.54) | (72.93) | (81.73) | (63.99) | (51.85) | (66.26) |
| Median | 352.0 | 341.0 | 367.0 | 358.0 | 283.0 | 338.0 |
| Minimum value, | 299, 426 | 227, 431 | 243, 508 | 237, 414 | 207, 364 | 207, 508 |
| maximum value | | | | | | |
| Changes over | | | | | | |
| baseline at last | | | | | | |
| visit (on day 6) | | | | | | |
| Number of | 12 | 9 | 9 | 9 | 9 | 48 |
| subjects | | | | | | |
| Mean (standard | 3.3 | 11.3 | 22.1 | 15.1 | −13.4 | 7.4 |
| deviation) | (35.40) | (35.95) | (36.29) | (46.23) | (41.98) | (39.30) |
| Median | 6.5 | 22.0 | 19.0 | 9.0 | −20.0 | 9.0 |
| Minimum value, | −79, 56 | −48, 52 | −27, 88 | −34, 122 | −75, 58 | −79, 122 |
| maximum value | | | | | | |
| P value | 0.750 | 0.372 | 0.105 | 0.356 | 0.365 | |

Abbreviation: N = Number of subjects in safety analysis set.
P value is obtained from paired t test.
*P < 0.05.

TABLE 2.1
Changes of uric acid index before and after treatment with Compound I
Multiple-dose groups
Safety analysis set
Index: Uric acid (UA) (μmol/L)

| Visit | Placebo | Compound I 10 mg | Compound I 20 mg | Total |
|---|---|---|---|---|
| Statistics | (N = 6) | (N = 9) | (N = 9) | (N = 24) |
| Baseline | | | | |
| Number of | 6 | 9 | 9 | 24 |
| subjects | | | | |
| Mean (standard | 305.3 (53.16) | 314.4 (84.59) | 337.2 (50.38) | 320.7 (64.57) |
| deviation) | | | | |
| Median | 313.0 | 290.0 | 349.0 | 305.0 |
| Minimum value, | 223, 367 | 215, 452 | 259, 400 | 215, 452 |
| maximum value | | | | |
| Day 2 | | | | |
| Number of | 6 | 9 | 9 | 24 |
| subjects | | | | |
| Mean (standard | 329.2 (32.55) | 254.9 (74.41) | 221.6 (63.82) | 261.0 (73.54) |
| deviation) | | | | |
| Median | 330.0 | 248.0 | 216.0 | 244.5 |
| Minimum value, | 292, 372 | 155, 397 | 142, 373 | 142, 397 |
| maximum value | | | | |
| Changes over baseline | | | | |
| on day 2 | | | | |
| Number of | 6 | 9 | 9 | 24 |
| subjects | | | | |
| Mean (standard | 23.8 (37.49) | −59.6 (40.77) | −115.7 (55.40) | −59.8 (70.69) |
| deviation) | | | | |
| Median | 12.0 | −50.0 | −142.0 | −53.5 |
| Minimum value, | −23, 76 | −135, 6 | −184, −26 | −184, 76 |
| maximum value | | | | |
| P value | 0.180 | 0.002* | <0.001* | |

Abbreviation: N = Number of subjects in safety analysis set.
P value is obtained from paired t test.
*P < 0.05.

TABLE 2.2
Changes of uric acid index before and after treatment with Compound I
Multiple-dose groups
Safety analysis set
Index: Uric acid (UA) (μmol/L)

| Visit | Placebo | Compound I 10 mg | Compound I 20 mg | Total |
|---|---|---|---|---|
| Statistics | (N = 6) | (N = 9) | (N = 9) | (N = 24) |
| Days 4-6 | | | | |
| Number of subjects | 6 | 9 | 9 | 24 |
| Mean (standard | 338.2 (48.30) | 201.8 (58.87) | 158.0 (51.31) | 219.5 (88.91) |
| deviation) | | | | |
| Median | 335.0 | 204.0 | 140.0 | 199.5 |
| Minimum value, | 271, 392 | 119, 307 | 105, 267 | 105, 392 |
| maximum value | | | | |
| Changes over baseline on | | | | |
| days 4-6 | | | | |
| Number of subjects | 6 | 9 | 9 | 24 |
| Mean (standard | 32.8 (31.24) | −112.7 (57.68) | −179.2 (54.55) | −101.3 (97.59) |
| deviation) | | | | |
| Median | 40.0 | −118.0 | −175.0 | −118.5 |
| Minimum value, | −24, 68 | −234, −35 | −260, −108 | −260, 68 |
| maximum value | | | | |
| P value | 0.050* | <0.001* | <0.001* | |

Abbreviation: N = Number of subjects in safety analysis set.
P value is obtained from paired t test.
*P < 0.05.

TABLE 2.3
Changes of uric acid index before and after treatment with Compound I
Multiple-dose groups
Safety analysis set
Index Uric acid (UA)(μmol/L)

| Visit | Placebo | Compound I 10 mg | Compound I 20 mg | Total |
|---|---|---|---|---|
| Statistics | (N = 6) | (N = 9) | (N = 9) | (N = 24) |
| Day 11 | | | | |
| Number of subjects | 6 | 9 | 9 | 24 |
| Mean (standard | 320.7 (57.16) | 325.1 (67.57) | 341.8 (58.10) | 330.3 (59.65) |
| deviation) | | | | |
| Median | 337.0 | 314.0 | 323.0 | 319.5 |
| Minimum value, | 220, 370 | 222, 439 | 262, 460 | 220, 460 |
| maximum value | | | | |
| Changes over baseline on | | | | |
| day 11 | | | | |
| Number of subjects | 6 | 9 | 9 | 24 |
| Mean (standard | 15.3 (34.69) | 10.7 (31.86) | 4.6 (43.38) | 9.5 (35.89) |
| deviation) | | | | |
| Median | 6.0 | 15.0 | 2.0 | 5.0 |
| Minimum value, | −19, 79 | −52, 60 | −52, 67 | −52, 79 |
| maximum value | | | | |
| P value | 0.328 | 0.345 | 0.761 | |

Abbreviation: N = Number of subjects in safety analysis set.
P value is obtained from paired t test.
* P < 0.05.

TABLE 2.4
Changes of uric acid index before and after treatment with Compound I
Multiple-dose groups
Safety analysis set
Index Uric acid (UA)(μmol/L)

| Visit | Placebo | Compound I 10 mg | Compound I 20 mg | Total |
|---|---|---|---|---|
| Statistics | (N = 6) | (N = 9) | (N = 9) | (N = 24) |
| Last visit (day 14) | | | | |
| Number of subjects | 6 | 9 | 9 | 24 |
| Mean (standard | 302.3 (47.53) | 326.9 (74.58) | 378.4 (37.55) | 340.1 (62.71) |
| deviation) | | | | |
| Median | 304.5 | 337.0 | 376.0 | 353.5 |
| Minimum value, | 225, 359 | 219, 422 | 309, 440 | 219, 440 |
| maximum value | | | | |
| Changes over baseline at | | | | |
| last visit (on day 14) | | | | |
| Number of subjects | 6 | 9 | 9 | 24 |
| Mean (standard | −3.0 (57.31) | 12.4 (37.22) | 41.2 (44.32) | 19.4 (47.06) |
| deviation) | | | | |
| Median | −2.5 | 6.0 | 40.0 | 14.5 |
| Minimum value, | −73, 92 | −45, 58 | −11, 135 | −73, 135 |
| maximum value | | | | |
| P value | 0.903 | 0.345 | 0.024* | |

Abbreviation: N = Number of subjects in safety analysis set.
P value is obtained from paired t test.
*P < 0.05.

### Example 3. Comparison of Compound I with Other Uric Acid-Reducing Drugs

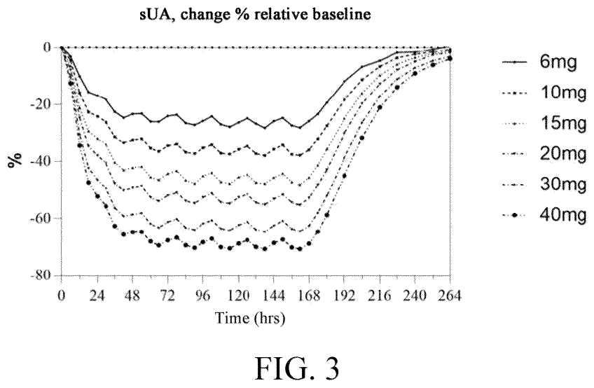

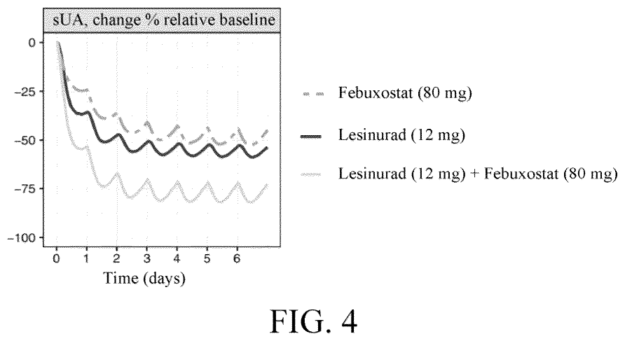

According to the clinical trial data of Example 2, based on the results of uric acid changes compared with the baseline after two different doses (10 mg and 20 mg) of Compound I administered once a day for consecutive 7 days, the present inventor predicted the uric acid-reducing effect of Compound I at four different doses (6 mg, 15 mg, 30 mg, and 40 mg) administered once a day for consecutive 7 days using a PK/PD model. The specific results are shown in FIG. 3. Compared with the disclosed data with lesinurad and febuxostat single drug and in combination thereof (see Jacob Leander, Journal of Pharmacokinetics and Pharmacodynamics https://doi.org/10.1007/s10928-021-09747-y, and FIG. 4), Compound I shows a better uric acid level reducing effect than lesinurad and febuxostat single drug at the same or lower doses, and even achieves a uric acid level reducing effect comparable to that of lesinurad and febuxostat in combination thereof.

### Example 4. Comparative Test of Compound I with Other Uric Acid-Reducing Drugs

This example aims to compare the effect of compound I with other uric acid-reducing drugs in reducing uric acid level. The design scheme of the animal experiment is as follows.

| Test drug | Compound I | | | | |
|---|---|---|---|---|---|
| Positive control | febuxostat, roflumilast, and apremilast, | | | | |
| Vehicle control | TBD | | | | |
| Test animals | UOX mice, SPF grade | | | | |
| | 50% male and 50% female | | | | |
| | 5-6 weeks old, | | | | |
| | 40 mice were purchased, and 30 animals were administered, 6 B6 mice | | | | |
| | were additionally purchased and used as a wild-type control | | | | |
| Test method | The animals were administered with the drugs after grouping and uric | | | | |
| | acid was detected daily | | | | |
| | | Dose | Concentration | Volume dosed | Number of |
| | Group | (mg/kg) | (mg/mL) | (mL/kg) | animals |
| Animal | Wild-type | — | — | — | 6 |
| grouping and | control group | | | | |
| dose of | Model | — | — | — | 6 |
| administration | control group | | | | |
| | Roflumilast | 0.1 | TBD | TBD | 6 |
| | Positive | | | | |
| | control group | | | | |
| | (0.5 mg QD) | | | | |
| | Apremilast | 6.3 | | | |
| | Positive | | | | |
| | control group | | | | |
| | Febuxostat | 8.3 and 16.7 | | | |
| | Positive | | | | |
| | control group | | | | |
| | Low-dose | 2.1 | TBD | TBD | 6 |
| | test drug | | | | |
| | group | | | | |
| | Medium-dose | 4.2 | TBD | TBD | 6 |
| | test drug | | | | |
| | group | | | | |
| | High-dose | 8.3 | TBD | TBD | 6 |
| | test drug | | | | |
| | group | | | | |

| | |
|---|---|
| Route of | Intragastric |
| administration | |
| Administration | Once a day, for consecutive 4 weeks |
| frequency and | |
| times | |
| Test indexes | 1) Clinical observation: twice a day. |
| | 2) Body weight and food intake: once before administration, and twice a |
| | week after administration |
| | 3) Blood uric acid: once before first administration, and once every 1, 2, |
| | 4, and 8 hours after administration, and then twice a week |
| | 4) Renal function and pathology: animals were euthanized after the last |
| | administration, and blood was taken to meeasure serum creatinine and |
| | urea nitrogen. The kidney was immobilized and stained with HE to |
| | evaluate pathological and histological changes. |

It is expected that the effect of Compound I in reducing uric acid level is comparable to or better than other uric acid reducing drugs.

1. A method of reducing uric acid level, preventing an increased uric acid level, or treating, preventing, or alleviating a disease, disorder, or condition associated with an increased uric acid level in an individual, wherein the method comprises administering a compound of Formula (I) to the individual:

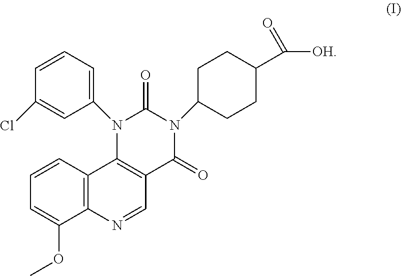 [SMILES: COc1cccc2c1ncc1c(=O)n(C3CCC(C(=O)O)CC3)c(=O)n(-c3cccc(Cl)c3)c12]

2. (canceled)

3. The method according to claim 1, wherein the compound of Formula (I) has a structure of Formula (Ia) or Formula (Ib):

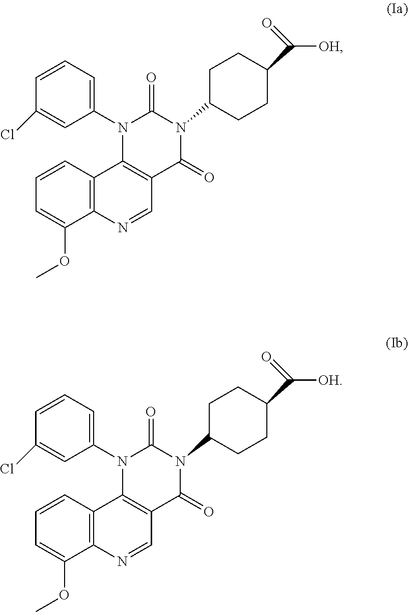

4. The method according to claim 3, wherein the compound of Formula (I) has a structure of Formula (Ia):

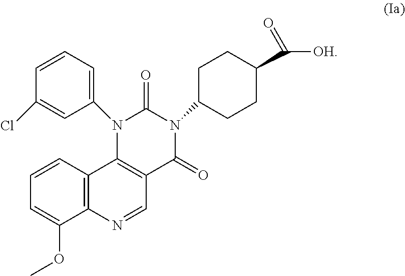

5. The method according to claim 1, wherein the disease, disorder, or condition associated with an increased uric acid level is selected from the group consisting of: gout, hyperuricemia, high uric acid level that does not reach the level usually diagnosed as hyperuricemia, renal dysfunction, urolithiasis, renal calculus, renal failure, diabetes, diabetes associated conditions, insulin resistance, metabolic syndrome, hypertension, hypothyroidism, hyperparathyroidism, obesity, inflammation, muscle spasm, local swelling, joint pain, myocardial infarction, arthritis, tumor lysis syndrome, cognitive impairment, lead poisoning, psoriasis, sarcoidosis, non-alcoholic fatty liver disease, non-alcoholic fatty hepatitis, cardiovascular diseases, atherosclerosis, and transplants of blood, bone marrow or solid organs.

6. The method according to claim 5, wherein the hyperuricemia is primary hyperuricemia or secondary hyperuricemia.

7. The method according to claim 6, wherein the secondary hyperuricemia comprises hyperuricemia related to drugs or hyperuricemia related to various medical conditions.

8. The method according to claim 1, wherein the uric acid level is the serum uric acid level.

9. The method according to claim 1, wherein the individual is further administrated one or more additional uric acid-reducing drugs.

10. The method according to claim 9, wherein the additional uric acid-reducing drug is selected from the group consisting of: xanthine oxidase inhibitors, uricosuric agents, urate transporter-1 inhibitors, uricase, and inhibin.

11. The method according to claim 9, wherein the additional uric acid-reducing drug is selected from the group consisting of: allopurinol, febuxostat, lesinurad, benzbromarone, roflumilast, apremilast, sodium bicarbonate, potassium sodium hydrogen citrate, potassium sodium citrate, non-steroidal anti-inflammatory drugs, glucocorticoids, and colchicine.

12. The method according to claim 1, wherein the individual is a mammal.

13. The method according to claim 12, wherein the individual is human.

14. The method according to claim 1, wherein before the medicament is administered to the individual, twice fasting serum uric acid levels in the individual on different days are higher than 420 μmol/L.

15. The method according to claim 1, wherein the medicament is administered orally, subcutaneously, intravenously, intramuscularly or intraperitoneally.

16. The method according to claim 1, wherein the medicament comprises no more than 5 mg, 10 mg, 15 mg, 20 mg, 25 mg, 30 mg, 35 mg, 40 mg, 45 mg, or 50 mg of the compound of Formula (I) or a pharmaceutically acceptable salt thereof.

17. The method according to claim 1, wherein the medicament is administered to the individual once a day, twice a day, or three times a day.
